(19) United States
(12) Patent Application Publication
(10) Pub. No.: US 2003/0180957 A1
(43) Pub. Date: Sep. 25, 2003

(54) Target and method

(75) Inventors: Jens-Oliver Koopmann, Cambridge (GB); Jonathan Michael Blackburn, Cambridge (GB)

Correspondence Address: TESTA, HURWITZ & THIBEAULT, LLP, HIGH STREET TOWER, 125 HIGH STREET, BOSTON, MA, 02110, US

(73) Assignee: Sense Proteomic Limited

(21) Appl. No.: 10/325,477

(22) Filed: Dec. 20, 2002

(30) Foreign Application Priority Data
Dec. 21, 2001 (GB) GB 0130747.9
Jul. 15, 2002 (GB) GB 0216387.1

(51) Int. Cl. G01N 33/00

(52) U.S. Cl. 436/56; 436/86; 436/173

(57) ABSTRACT

The invention relates to a probe for the analysis of one or more proteins by laser desorption/ionisation mass spectrometry. The proteins include a tag, which in turn include a biotin group. The probe includes at least one surface including one or more of streptavidin, avidin or neutravidin molecules to bind the biotin group to the surface. The proteins can also carry a BCCP tag. The probe can form a protein array of two or more proteins at know locations on the surface of a chip. The invention also relates to methods of analysis by laser desorption/ionisation mass spectrometry using the probe. A scalable MALDI target volume sample volume loading kit and methods for its use are provided herein.

### REFERENCE TO RELATED APPLICATIONS

This application claims priority to British applications, GB 0130747.9, filed Dec. 21, 2001, and GB 0216387.1, filed Jul. 15, 2002. The disclosures of each of these patent applications are incorporated by reference herein.

### BACKGROUND OF THE INVENTION

Hutchins and Yip, 1993 introduced affinity-capture of proteins on MALDI-TOF sample carrier. [T. William Hutchens and Tai-Tung Yip, 1993 Rapid Communication in Mass Spectrometry, 7,576-580.] This work presented a starting point for the development of affinity capture matrices on MALDI-TOF sample carriers. Hutchins and Yip captured lactoferrin with DNA-agarose from preterm infant urine. The agarose beads with affinity captured protein were loaded on the MALDI target, overlaid with energy absorbing matrix molecules and a good quality mass spectrum was acquired, whereas the unfractionated urine resulted in a poor mass spectrum due to signal suppression caused by salts and other proteins present in a complex sample as urine.

The technology presented by Hutchins and Yip was slightly improved by Nelson et al., 1995 who describe a mass spectrometric immunoassay (MSIA) that can identify myotoxin and mojave toxin from blood samples. [Randall W. Nelson, Jennifer R. Krone, Allan R. Bieber, and Peter Williams. (1995) Analytical Chemistry 67, 1153-1158.] Affinity purified rabbit serum against the two toxins was conjugated to protein A agarose and the affinity matrix was then used to screen whole blood samples for the presence of myotoxin or mojave toxin. The major improvement described herein is the direct elution of the affinity-captured ligand onto the MALDI sample carrier. This enables to achieve higher mass accuracy in the MALDI process due to a homogenous flat layer of crystals compared to the heterogeneous surface preparation of Hutchins and Yip.

Brockman and Orlando described in 1995 the derivatisation of a MALDI surface with a goat antibiotin antibody. [Adam H. Brockman and Ron Orlando. (1995) Analytical Chemistry 67, 4581-4585.] The covalent attachment of the antibody was mediated via dithiobissuccinimide, which binds proteins at their free amine group. The antibody was used to analyze various complex mixtures of proteins that were spiked with biotinylated proteins. MALDI spectra of the complex mixture and the affinity purified samples demonstrated that the antibiotin antibody could selectively bind biotinylated proteins and could therefore enrich these molecule species. In a further example this group coupled an anti-lysozyme antibody to the MALDI target and probed it with 1 microliter of teardrop. The antibody probed affinity capture surface was able to resolve a lysozyme signal at 15 kDa whereas the unfractionated tear drop sample on a normal MALDI target surface resulted in a very poor and broad signal. This publication demonstrated for the first time the affinity capture of proteins via antibody on a MALDI target.

Liang et al., 1998 have proposed an alternative way of immobilizing antibodies using nitrocellulose as a scaffold on a MALDI substrate. [Xiaoli Liang, David Lubman, David T. Rossi, Gerald D. Nordblom, and Charles M. Barksdale. (1998) Analytical Chemistry 70, 498-503.] A polyclonal antibody against SNX-111 was allowed to bind to nitrocellulose on the MALDI target, excess of antibody was rinsed with water, serum previously spiked with the antigen was applied on top of the immobilized antibody for 20 minutes and unbound ligands were washed away with ample water. The authors could demonstrate that the antigen was recovered from a mixture of serum proteins at high sensitivity. Liang et al. paved the road for a more general immobilization strategy of proteins on MALDI targets.

Davies et al. described profiling of amyloid beta peptide variants using a commercialized preactivated MALDI target from Ciphergen Biosystems that is ready to use for protein coupling. [Huw Davies, Lee Lomas and Brian Austen, (1999) Biotechniques 27, 1258-1261]. The chip has 8 positions for covalent binding of antibody on the chip surface. Based on the coupling chemistry the orientation of the antibody on the chip surface is random. The immobilized antibody can be used to capture antigens from biological fluids such as cell culture supernatants (Davies et al., 1999, and Diamond et al., 2001), tear drops (Brockman and Orlando, 1995), and serum (Nelson et al., 1995). Antigens captured on the MALDI surface can be separated from other biological molecules with a few washing steps followed by the application of a energy absorbing matrix. The matrix molecules dissociate the antigen from the antibody and can be analyzed by MALDI TOF. Davies et al. analyzed amyloid beta peptide variants in the range of 2000 to 6000 Dalton with an accuracy of 1 Dalton.

### SUMMARY OF THE INVENTION

The invention generally includes a probe for analysis of one or more proteins by laser desorption/ionisation mass spectrometry. Generally, proteins includes a tag which in turn further includes a biotin group. According to the invention, a probe includes at least one surface further including one or more streptavidin, avidin or neutravidin molecules that bind the biotin group to the surface.

In one embodiment of the invention, a probe further includes one or more proteins carrying a tag bound via a biotin group to one or more streptavidin, avidin or neutravidin molecules present on the surface of the probe. In related embodiments, one or more streptavidin, avidin or neutravidin molecules are present on the surface of a probe with a protein repellent coating on the surface. Also in related embodiments, a probe further includes a protein repellent coating including polyethylene biotin conjugated poly-L-lysine. In other embodiments, a tag is a BCCP tag or an Avi-tag (biotinylated peptide). In related embodiments, a probe further includes two or more proteins attached via a biotin group at known locations on the surface of the chip to form a protein array.

The invention also includes a method of analysis by laser desorption/ionisation mass spectrometry. Methods of the invention generally include the steps of: providing a probe including at least one surface including one or more streptavidin, avidin or neutravidin molecules; bringing the probe into contact with one or more proteins including a tag which in turn carries a biotin group under conditions allowing the biotin group to bind to at least one of the streptavidin, avidin or neutravidin molecules; and, performing laser desorption/ionisation mass spectrometry on the proteins on the surface of the probe. Methods of the invention also include the alternative steps of: bringing the probe into contact with one or more proteins including a tag which in turn carries a biotin group under conditions allowing the biotin group to bind to at least one of the streptavidin, avidin or neutravidin molecules; and, removing unbound molecules from the probe. In related embodiments, one or more proteins are contained in a mixture of tagged and untagged proteins.

According to the invention, methods of identifying a protein on the surface of the probe also include the steps of: determining the mass of the protein molecule; performing a digestion upon a replicate sample of the protein on a further probe or probe surface; performing laser desorption/ionisation mass spectrometry on the peptides to identify the protein. In addition, methods of analysing the function of a protein on the surface of the probe and a molecule interacting with the protein also include the steps of: bringing a protein on the probe surface into contact with one or more test molecules; removing unbound test molecules from the probe surface; and, performing laser desorption/ionisation mass spectrometry on the protein and any bound molecule to determine the identity of the protein and/or test molecule.

Furthermore, methods of analysing the function of a protein also include the steps of: bringing a protein on the probe surface into contact with one or more test substrates; and, performing laser desorption/ionisation mass spectrometry on the protein and test substrates to determine the presence and/or identity of products of catalysis of the test substrates by the protein.

Methods of analysing the folded structure of a protein also include the steps of: determining the mass of the protein molecule; performing hydrogen/deuterium exchange on solvent accessible amide groups on a replicate sample of the protein on a further probe or probe surface; performing laser desorption/ionisation mass spectrometry on the protein to determine the mass of the protein; bringing the replicate sample of the protein on a further probe or probe surface into contact with a denaturant to unfold the protein; performing hydrogen/deuterium exchange on solvent accessible amide groups on the protein sample on the surface of the probe; performing laser desorption/ionisation mass spectrometry on the protein to determine the mass of the protein; and, comparing the information obtained from the steps to determine the folded structure of the protein.

In certain embodiments, a probe according to the invention carries a protein array. Also in certain embodiments, an array consists of individual proteins present at at least 96, 384, 1536 or 10,000 discrete locations on the probe surface.

The invention also includes a MALDI target sample volume loading device including a sample loading means, and a MALDI target holding means, where the sample loading means includes a plurality of apertures suitable for loading samples on to a MALDI target (when present) which is held adjacent to one surface of the sample loading means by way of releasable engagement between the sample loading means and the MALDI target holding means. Also, in related embodiments, the invention includes a MALDI target sample volume loading device where the sample loading means and the MALDI target holding means releasably engage through interlocking features present on each means. In other embodiments, the invention also includes a MALDI target sample volume loading device where a plurality of apertures suitable for loading samples on to a MALDI target are present as a regularly spaced array of apertures.

Also in related embodiments, a MALDI target sample volume loading device includes an array of apertures that include 384 apertures arranged in a 16×24 array. Also in related embodiments of the invention, a MALDI target sample volume loading device includes a sample loading means further including a liquid repellent layer on the surface adjacent to a MALDI target (when present). In certain embodiments of the invention, a MALDI target sample volume loading device includes a liquid repellent layer including a silicon layer. The invention also includes a MALDI target sample volume loading device that is provided in FIGS. 8, 9, 10 or 11.

In certain embodiments of the invention, a probe is brought into contact with one or more proteins including a tag by the use of a target sample volume loading device according to the methods described herein. Accordingly, methods of the invention also include steps that are carried out in one or more wells formed by a target sample volume loading device as provided, for example, in the sample loading devices described herein.

In certain embodiments, the invention also includes a MALDI target adaptor device suitable for locating one or more glass microscope slides on a MALDI target (when present), where the glass slides include a conductive coating, a protein resistant surface and a capture surface present on the protein resistant surface. In related embodiments, the invention includes a MALDI target adaptor device having a conductive coating including gold. Also in certain embodiments, the invention includes a MALDI target adaptor device having a capture surface including neutravidin, avidin or streptavidin. In certain embodiments, the invention also includes a MALDI target adaptor device that is suitable for locating four glass microscope slides on a MALDI target. Furthermore, in certain embodiments, the invention includes a MALDI target adaptor device including a MALDI target that is a Brucker Daltonics™ MALDI target. In certain embodiments, the invention also includes a MALDI target adaptor device that is provided in FIG. 12. Furthermore, in certain embodiments, the invention includes a probe mounted on a MALDI target adaptor device as described herein.

As discussed herein, proteins have a great diversity in their chemical nature and it is not trivial to immobilize them on a surface without losing or reducing their biological activity. The Inventors have developed new proteomic technologies to address these issues. In our approach towards proteomics we extract the mRNA of cells and create cDNA libraries. Individual cDNA libraries are expressed in heterologous hosts for example Escherichia coli, Aspergillus niger, Pichia pastoris or Spodoptera frugiperda (Sf9). During the cloning procedure COVET technology, as described in WO 01/57198, can be used to add a sequence tag to each protein. The “tag” sequence when fused to the target proteins provides a means to capture each fusion protein with exquisite specificity, thorough the same interaction in each case. Affinity tags are a convenient method of purification and immobilisation of recombinant proteins. Hexahistidine tags (6aa; Qiagen, Roche), Escherichia coli maltose binding protein (MBP, 300aa; New England Biolabs) and Schistosoma japonicum glutathione-S-transferase (GST, 220 aa; Amersham Pharmacia Biotech, Novagen) are effective, but have the disadvantage that heterologous host proteins interact with the affinity matrices used for purification of fusion proteins. This results in results in impure protein preparations and an additional clean up step is often required. Additionally, the relatively weak affinity of these proteins for their ligands results in dissociation, or “leaching” of the fusion proteins from surfaces to which they are immobilised. Such reversible interactions are exploited during resin-based purifications on resins in column or batch formats where, because of the high local concentrations of ligand, dissociated proteins rapidly rebind, yet are rapidly competitive displaced by free ligand. However, immobilisation of proteins to planar surfaces such as microtiter plates, microarrays (biochips) or targets for MADLI analysis, requires that they remain bound and do not leach from the substrate during storage and use. As such, lower affinity tags as used for purification (e.g. MBP, GST and hexahistidine tags) are suboptimal. Frequently, covalent immobilisation strategies are employed such as coupling of purified proteins via surface lysine residues to amine-reactive chemical groups. This is generally accepted to result in reduced activity of the protein. Biotin can be attached chemically to proteins (e.g. using NHS-activated biotin), or via genetically fused protein domains which are biotinylated in vivo. The “PinPointTM” vectors from Promega are designed to facilitate the creation of fusions to the biotin carboxyl carrier protein (BCCP) from Propionibacterium freudenreichii shermanii. This system allows the production of BCCP-protein fusions capable of being biotinylated either in vivo or in vitro by biotin ligase, allowing one to use the highly specific biotin—streptavidin interaction for surface capture. In addition to the BCCP domain phage display selected short peptides capable of being biotinylated on a lysine residue have been commercialised by Avidity Inc. and are the subject of U.S. Pat. No. 5,932,433.

A new approach is described herein whereby the E. coli BCCP is fused either N- or C-terminally to a protein partner, thus permitting orientated immobilization of the fusion protein to microarray and MALDI compatible surfaces derivatised with avidin, streptavidin or neutravidin. The Inventors have determined that use of a tag derived from the biotin carboxyl carrier protein (BCCP) of acetyl-CoA carboxylase of E. coli or a peptide sequence that can be biotinylated in vivo such as “Avi-Tag” is particularly suited to the attachment and capture of proteins to surfaces, in particular, the surfaces of targets/probes for use in laser desorption/ionisation mass spectrometry.

BCCP may be biotinylated in vivo and/or in vitro to allow capture by streptavidin or avidin or neutravidin on a surface. The biotin-streptavidin and biotin-avidin interactions are some of the highest affinity non-covalent interactions known, with equilibrium dissociation constants of 10-15M, which is several orders of magnitude higher affinity than the MBP-amylose, GST-glutathione, or hexahistidine-Ni²⁺ interactions. The fast on-rate of the streptavidin-biotin interaction means that proteins with low stability can be captured without needing to be incubated with the capture surface for long periods of time whilst the femtomolar K+ means that a million-fold lower fusion protein concentration is required for surface capture compared to an interaction with a nanomolar KD.

One major advantage of putting the same sequence tag on each protein is that it enables the parallel processing of a great number of different proteins on a protein array. The biotinylated TAG sequence can be recognised by, for example, a PEG-PLL-biotin neutravidin-coated MALDI target surface (see FIG. 7).

The Inventors have found that, compared to other affinity tags, immobilisation of proteins as BCCP fusions aids maintenance of the native folded state of the fusion proteins where immobilisation onto the solid surface is specifically via the biotin moiety of the BCCP domain. In addition, the high affinity of the biotin-streptavidin interaction, coupled with the protein-repellant nature of the surface coating, enables stringent washing of the surface after capture of the BCCP fusion proteins in order to remove salt, detergents, proteins, or other biological macromolecules such as nucleic acids or lipids that are not specifically bound to the surface. These features of proteins immobilised as BCCP fusions on protein-repellant surfaces enable the high throughput functional analysis of arrays of immobilised proteins by, for example, MALDI mass spectrometry methods. The types of functional analysis that are enabled include determination of the identity of the each protein in the array, determination of the folded state of each protein in the array, and determination of the interactions between each protein in the array and a molecule, or mixture of molecules, of interest.

Thus in a first aspect the invention provides a probe for analysis of one or more proteins by laser desorption/ionisation mass spectrometry, wherein said proteins comprise a tag which in turn comprises a biotin group and wherein said probe comprises at least one surface comprising one or more streptavidin, avidin or neutravidin molecules that bind said biotin group to said surface.

As defined herein a probe is a support which is capable of acting as a target in analysis by laser desorption/ionisation mass spectrometry, for example matrix assisted laser desorption/ionisation (MALDI).The probe carries the analytes, for example proteins, during such processes and interacts with the repeller lens of the ion-optic assembly found in laser desorption/ionisation time-of-flight (TOF) mass spectrometers of the art, such that the analytes are converted to gaseous ions to permit analysis. For example, the probes of the invention may be derived from targets for MALDI analysis as known in the art, which are treated such that streptavidin, avidin or neutravidin molecules are present on the probe surface and bind biotinylated proteins for subsequent analysis. For example, conventional glass or gold MALDI targets may be used.

As defined herein a tag which in turn comprises a biotin group is an amino acid tag such as a biotinylated protein domain, for example a BCCP tag or a biotinylated peptide for example an “Avi-Tag”, present in the sequence of a protein of interest which is capable of, or has undergone, conjugation with biotin. Alternatively domains derived from proteins other than BCCP or peptides other that Avi-Tag can be used provided that they are capable of being biotinylated when forming part of the protein or library of proteins of interest. Preferably, the protein of interest has been expressed in a host cell and the conjugation has taken place in vivo in the same host cell. In this situation, the protein of interest can advantageously be purified away from the other components of the host cell lysate on the target once it is bound. The high affinity of the binding between the tagged protein and the target probe permits washing of the target to remove proteinaceous and other components, e.g. salts, that would otherwise interfere with subsequent mass spectrometry analysis. The high affinity of the binding between the probe and the tag provided by the invention allows washing of the probe at high levels of stringency.

Whilst streptavidin, avidin or neutravidin molecules are the preferred means for attaching the tagged proteins to the target, naturally occurring or synthetic variants of these molecules, or other unrelated molecules, which also have a similar affinity for biotin are considered to be within the scope of the invention. Preferably, streptavidin, avidin or neutravidin molecules are attached to or are also present on a surface of the probe via or with a protein repellent coating on said surface. The coating can comprise one or more biotin molecules for example, biotin derivatised poly-L-lysine grafted polyethylene glycol co-polymers PEG-PLL-Biotin. Conventional methods known in the art involving, for example, chemical coupling or physical adsorption, may be used to attach streptavidin, avidin or neutravidin to the target surface directly or via attachment to biotin which itself is attached to the target surface by such methods.

As an example, the immobilisation of proteins on the MALDI target can be a three step process. The protein repellent polyethylene conjugated poly-L-lysine biotin (PEG-PLL-Biotin) Ruiz-Taylor et al., 2001 is first coated on MALDI glass or gold surfaces. In a second coating step the affinity capture matrix is overlaid with neutravidin and the surface is ready to immobilize biotinylated proteins. [L. A. Ruiz-Taylor, T. L. Martin, F. G. Zaugg, K. Witte, P. Indermuhle, S. Nock, and P. Wagner. 2001 PNAS, 98, 852-857.] In the third step the biotinylated BCCP fusion protein is added to the surface. The BCCP fusion protein can be applied to the surface as a crude mixture or as a purified protein. The capture of the biotinylated BCCP fusion protein on the PEG-PLL-Biotin-neutravidin surface is highly specific. Nonbiotinylated proteins, DNA, RNA, small molecules and salts can be washed with a detergent containing buffer followed by a desalting step to achieve the best conditions for the MALDI process. [Michael Karas and Franz Hillenkamp. (1988) Analytical Chemistry 60, 2301-2303.] Advantages of this procedure compared with current technologies are the defined orientation of the BCCP-fusion protein, very specific recognition of the fusion protein, maximum biological activity of the immobilized protein, minimized non-specific binding, very high protein density and homogenous distribution of the fusion protein on the affinity surface. The Inventors have shown that the BCCP tag can be fused to a protein on the N- or at the C-terminus, without affecting these properties. By comparison amine coupling reagents could react with the N-terminus and with any lysine in a protein. This amine coupling potentially results in hundreds of different orientations of the protein on the target or array of proteins on the target including multipoint attachment (see FIG. 7). The high specificity of the BCCP and neutravidin interaction enables the protein to be delivered in a complex mixture with other biological macromolecules for example as a cell lysate. The sample can be easily cleaned up by standard washing and desalting procedures. By keeping the immobilization chemistry the same in every case it is easy to automate the protein array production as well as creating standard washing procedures rather than protein or chip specific washing procedures. The noncharged, hydrophobic, biotinylated PEG-PLL-biotin surface minimizes the non-specific binding of proteins and other biological macromolecules to the surface. [Emanuele Ostuni, Robert G. Chapman, R. Erik Holmlin, Shuichi Takayama, and George M. Whitesides. 2001. Langmuir, 17, 6336-6343.] One feature of protein arrays fabricated in this manner is the homogeneous protein deposition on the PEG-PLL-biotin surface. This has important implications for the overall performance of the MALDI process since it results in a homogeneous distribution of the protein- or peptide-containing crystals that are formed after the array is overlaid with matrix. The consequence of this homogenous distribution of said crystals is that within any single area containing an immobilised protein, every co-ordinate interrogated by a MALDI laser source gives rise to an equivalent mass spectrum, thus removing the need to search for a “sweet spot” which is currently required in the art and greatly increasing the speed and ease of automated MALDI spectra acquisition.

In a second aspect of the invention the invention provides a probe which further comprises one or more proteins carrying a BCCP tag bound via a biotin group to one or more streptavidin, avidin or neutravidin molecules present on the surface of said probe. Thus probes to which proteins having a BCCP tag have been attached, and optionally treated either to wash away contaminant proteins or other molecules from the original sample that might interfere with later analysis, e.g. salts, are considered to be within the scope of the invention.

In a third aspect the invention provides a probe which comprises two or more proteins attached via a biotin group at known locations on the surface of the chip to form a protein array. As defined herein the term “protein array” relates to a spatially defined arrangement of one or more protein moieties in a pattern on a surface. The protein moieties will be attached to the surface through the biotin group attached to the protein domain derived (eg BCCP) tag or peptide tag linked to each protein. The array can consist of individual proteins present at at least 96, 384, 1536 or 10,000 discrete locations on said probe surface.

Thus, for example, each position in the pattern may contain one or more copies of:

a) a sample of a single protein type (in the form of a monomer, dimer, trimer, tetramer or higher multimer);

b) a sample of a single protein type bound to an interacting molecule (e.g. DNA, antibody, other protein); or

c) a sample of a single protein type bound to a synthetic molecule (e.g. peptide, chemical compound).

In a third aspect the invention provides a method of analysis by laser desorption/ionisation mass spectrometry comprising the steps of:

a) providing a probe comprising at least one surface comprising one or more streptavidin, avidin or neutravidin molecules;

b) bringing said probe into contact with one or more proteins comprising a tag which in turn comprises a biotin group under conditions allowing said biotin group to bind to at least one of said streptavidin, avidin or neutravidin molecules;

c) performing laser desorption/ionisation mass spectrometry on the proteins on the surface of the probe.

In one embodiment the method comprises the alternative steps of:

b)i) bringing said probe into contact with one or more proteins comprising a tag which in turn carries a biotin group under conditions allowing said biotin group to bind to at least one of said streptavidin, avidin or neutravidin molecules;

b)ii) removing unbound molecules from the probe

In a further embodiment the method is performed upon one or more proteins which are contained in a mixture of tagged and untagged proteins, for example a crude lysate from a culture of host cells.

In a further embodiment the method is a method for identifying a protein on the surface of the probe and which comprises the additional steps of:

d) determining the mass of the protein molecule

e) performing a digestion upon a replicate sample of said protein on a further probe or probe surface

f) performing laser desorption/ionisation mass spectrometry on the peptides resulting from step e) to identify said protein.

In a further embodiment the method is a method of analysing a protein on the surface of the probe and a molecule interacting with said protein and which comprises the additional steps of:

c) bringing a protein on the probe surface into contact with one or more test molecules;

d) removing unbound test molecules from the probe surface;

e) performing laser desorption/ionisation mass spectrometry on the protein and any bound molecule to determine the identity of the protein and/or test molecule.

In a further embodiment the method is a method of analysing the function of a protein and which comprises the additional steps of:

c) bringing a protein on the probe surface into contact with one or more test substrates

d) performing laser desorption/ionisation mass spectrometry on the protein and test substrates to determine the presence and/or identity of products of catalysis of said test substrates by the protein.

In a further embodiment the method is a method of analysing the folded structure of a protein and which comprises the additional steps of:

d) determining the mass of the protein molecule;

e) performing hydrogen/deuterium exchange on solvent accessible amide groups on a replicate sample of said protein on a further probe or probe surface;

f) performing laser desorption/ionisation mass spectrometry on the protein resulting from step e) to determine the mass of said protein;

g) bringing the replicate sample of said protein on a further probe or probe surface into contact with a denaturant to unfold said protein;

h) performing hydrogen/deuterium exchange on solvent accessible amide groups on the protein sample of step g) on the surface of the probe

i) performing laser desorption/ionisation mass spectrometry on the protein resulting from step h) to determine the mass of said protein;

j) comparing the information obtained from steps d), f) and i) to determine the folded structure of the protein.

In a fourth aspect the invention provides a scalable MALDI target sample volume loading kit, as claimed herein, useful in the methods of the invention. A problem encountered with MALDI targets is that such targets can take only very small amounts of liquid in each sample position. If higher volumes are required, for example on diluted samples that could not easily concentrated, it is desirable to increase amount of liquid on the MALDI surface.

The Inventors have devised a MALDI target sample volume loading device useful in the methods of the invention, and for example, for affinity capture of diluted proteins from protein mixtures. Conventional MALDI targets allow only the application of small amounts of liquid, which dry within minutes after application. This can become a problem if spotting several hundred samples. The MALDI target adapter described herein allows the spotting of larger volumes and delays drying out of the sample, whilst permitting purification and other biochemical steps (e.g. exposing proteins on a MALDI probe array to a potential ligand) to be carried out on the surface of the probe prior to MALDI analysis.

Current MALDI targets can be loaded only with 1-2 μl volumes per sample and are not well suited for biochemical interaction reactions. This would hinder, for example, the purification by capture on an MALDI probe affinity surface of recombinant proteins from E. coli, yeast, insect cells, mammalian cells which are expressed with a sequence tag (His tag, FLAG-tag, biotin-tag or any other tag). The device presented herein is a sample volume loading kit compatible with conventional MALDI targets. It can form, for example, 96/384/1536 separated wells on top of a MALDI target. The MALDI target can be sealed against the adapter with liquid repellent layer, for example a 2 mm silicon layer. The layer used is ideally chemically inert against aqueous solutions.

Thus the target loading device of this aspect of the invention may be employed in the methods described herein to bring the probes of the invention into contact with one or more proteins comprising a tag (for example, steps b and b(i) as described above).

Thus, in a fifth aspect, the invention provides probes and methods in which they are used, which accord with the invention and wherein the probe is brought into contact with with one or more proteins comprising a tag by the use of a target sample volume loading device according to the fourth aspect of the invention.

The target loading device may also be used to provide a well in which further treatment steps are performed on the spotted proteins on the probe (for example, ligand interaction experiments as mentioned above).

Thus, in a sixth aspect, the invention provides a method of the third aspect of the invention in which the alternative steps or additional steps described herein are carried out in one or more wells formed by a target sample volume loading device according to to the fourth aspect of the invention.

In a seventh aspect the invention provides an adaptor device for mounting protein arrays upon a MALDI target as claimed herein. At present protein arrays are commonly printed out on microscope glass whereas mass spectrometer MALDI targets are of the size of 122×86 mm. The Inventors have developed an adaptor device which comprises an interface to hold a plurality of solid substrate elements intended to carry a protein array (for example, glass microscope slides of 26×76 mm) on a MALDI target (for example a 122×86 mm Bruker Daltonics™ MALDI target). The microscope glass slide has a gold coating that confers conductivity to it. The glass slide is conductive and carries a protein resistant surface. The surface carries a capture surface, for example Neutravidin or Avidin or Streptavidin to capture biotinylated proteins, peptides, carbohydrates, DNA, RNA or non-biological homo-polymers or hetero-polymers.

The current technology in creating protein arrays on MALDI targets suffers from the lack of a spatial defined geometry and from the availability of purified proteins in order to populate the array. Protein arrays as they are described by X. Li, S. Mohan, W. Gu, N. Miyakoshi and D. J. Baylink. (2000) Biochimica et Biophysica Acta 1524, 102-109, and Shounyou Wang, Deborah L. Diamond, G. Michael Hass, Roger Sokoloff and Robert L. Vessela. (2001) International Journal of Cancer, 92, 871-876, lack a defined protein composition, spatial definition and protein loading is variable. The deposition does not occur via defined macromolecular interaction of binding partners, resulting in a random orientation of proteins on the array. Furthermore, the present immobilization strategies suffer from significant non-specific binding e.g. the capture surface is not protein repellent and so does not prevent non-specific binding (Brockman and Orlando, 1995 and Nelson et al., 1995). Current protein arrays on MALDI targets immobilize or capture proteins on the surface due to a variety of interactions. These interactions are often different for each protein and therefore it is very difficult to establish standard procedures for washing and incubation steps which are very important if high throughput is desired.

Proteins themselves are very diverse in their chemistry and lack a common motif for the immobilization of active proteins that fulfils the criteria of defined spatial distribution, retention of biological activity, defined loading density and defined orientation. A second feature of the protein array surface must be a protein repellent behavior to minimize non-specific binding to the surface. A commonly used feature to tether proteins on a surface is to immobilize them via an amine group (Brockman and Orlando, 1995, Davis et al., 1999), via coupled antibodies (Brockman and Orlando, 1995, Davies et al. 1999, Diamond et al., 2001), via hydrophobic interactions (Liang et al., 1998), via ionic interaction (Li et al, 2000), or affinity metal ion interactions [Vanitha Thulasiraman, Sandra L. McCutchen-Maloney, Vladimir L. Motin and Emilo Garcia. (2001) BioTechniques 30, 428-432, and Gunter E. M. Tovar, Thomas Schiestel, Christain Hoffmann and Jurgen Schmucker. (2001) Bioforum International, 5, 235-237]. All the methods listed above fail the criteria of immobilizing proteins in a defined orientation, spatial distribution, defined loading density and retaining maximum biological activity. Immobilizing proteins via an immobilized antibody has the highest specificity of the above mentioned methods, but the loading density of the ligand is reduced since the antibody is coupled in a random orientation on the MALDI target (Nelson et al., 1995). Secondly this approach is subject to the availability of an appropriate antibody.

Advantageously the methods of the invention are carried out upon multiple proteins in parallel on a probe which carries a protein array. This allows such methods to be carried out in a high-throughput fashion. The adaptor device of the invention allows multiple such protein arrays to be located on a MALDI target.

Thus in an eighth aspect the invention provides probes, and methods in which they are used, which accord with the invention and wherein the probe is mounted on a MALDI target adaptor device according to the fifth aspect of the invention.

Preferred features of each aspect of the invention are as defined for each other aspect, mutatis mutandis.

Further features, details, and embodiments of the invention will be apparent upon review of the following description of specific embodiments that follows. The use of the following description in accordance with the invention is provided below by way of example with reference to the accompanying drawings, in which:

### BRIEF DESCRIPTION OF THE DRAWINGS

FIG. 1 shows surface capture of neutravidin on a poly-L-lysine poly ethylene glycol-biotin (PLL-PEG-biotin) derivatised MALDI glass surface. The MALDI glass surface was previously coated with PLL-PEG-biotin for 1 hour in a humid chamber. Unbound PLL-PEG-biotin was washed away with 1 mM Tris-HCl pH 7.5 and 0.1% Triton X-100 and the surface was dried with 99.9996% nitrogen. 500 nanolitre of 0.5 mg/ml neutravidin was then overlaid on the PEG-PLL-biotin surface as well as on the blank glass surface. After one hour the whole MALDI target was washed in 1 mM Tris-HCl pH 7.5 and 0.1% Triton X-100 followed by a desalting step in 1 mM Tris-HCl pH 7.5. The MALDI target is then dried under nitrogen and an energy absorbing matrix is overlaid onto the MALDI surface. The analysis of the PEG-PLL-biotin surface probed with neutravidin is depicted above. The analysis of the glass surface without PEG-PLL-biotin coating showed no neutravidin signal at 14663 Dalton.

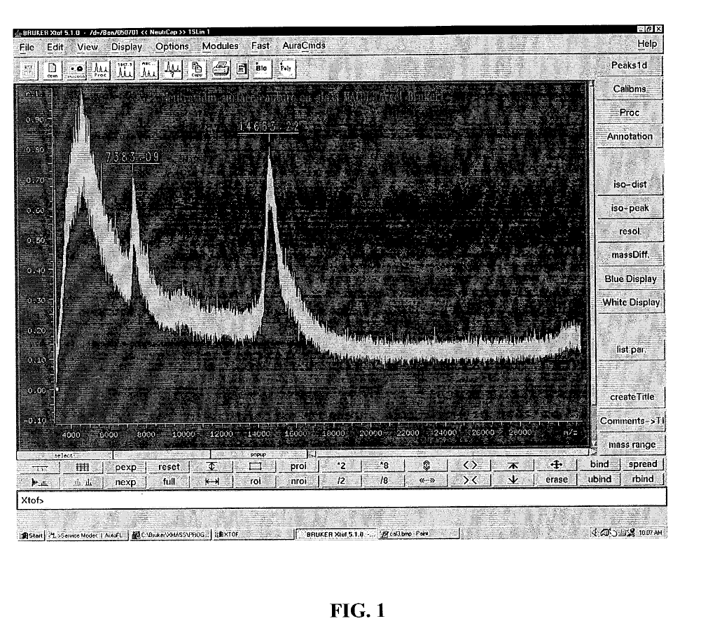

FIG. 2 shows neutravidin captured on a MALDI target coated with PEG-PLL-biotin washed with 1 mM Tris-HCl pH 7.5 and 0.1% Triton followed by a 1 mM Tris-HCl pH 7.5 wash. For the digestion 500 nl of 5 ng trypsin in 25 mM ammonium bicarbonate pH 7.5 is added. After one hour incubation at 37 C in a humid chamber the target is dried under nitrogen and a energy absorbing matrix is added and a mass spectrum was collected in the reflectron mode.

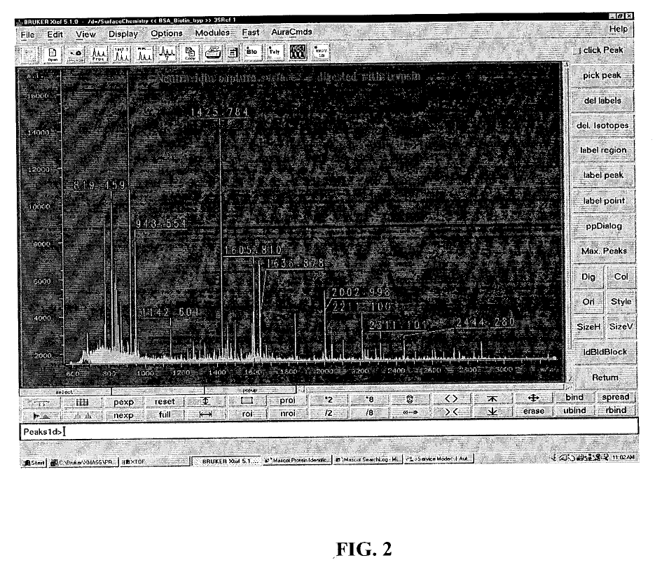

FIG. 3 shows biotinylated BSA specifically captured on a neutravidin coated MALDI target, digested with trypsin and a peptide spectrum was collected. The specificity of the surface capture was confirmed with the following experiments. BSA-Biotin was deposited on the MALDI target without Neutravidin or without the Biotin layer. BSA-Biotin was not detected in either case. Furthermore, binding of BSA-biotin to the Neutravidin coated surface is inhibited by the presence of free biotin. The database search results for BSA-biotin are presented in the appendix A.

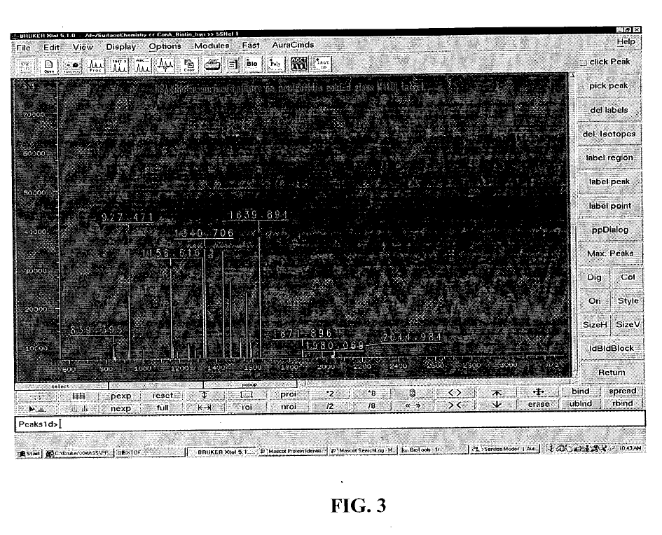

In the MALDI spectra peptide the peaks derived from Neutravidin and Trypsin are parsed out in the peak annotation to set the focus on secondary captured protein.

FIG. 4 shows biotinylated ConA was captured on a Neutravidin coated MALDI target surface and digested on the target. The ConA peptide spectrum is free of the Neutravidin peptide peaks, which were detected in the digest of the Neutravidin only surface. Neutravidin peaks: 819.46, 835.45, 919.52, 1425.78,1594.87,1837.89 and 2003.00 were absent in this case. The 919.52 peptide peak was detected in the BSA-Biotin digest FIG. 3 which was assigned to Neutravidin. This would imply that Neutravidin derived peptide peaks do not interfere with database searches. The database search results for Concanavalin A are presented in the appendix.

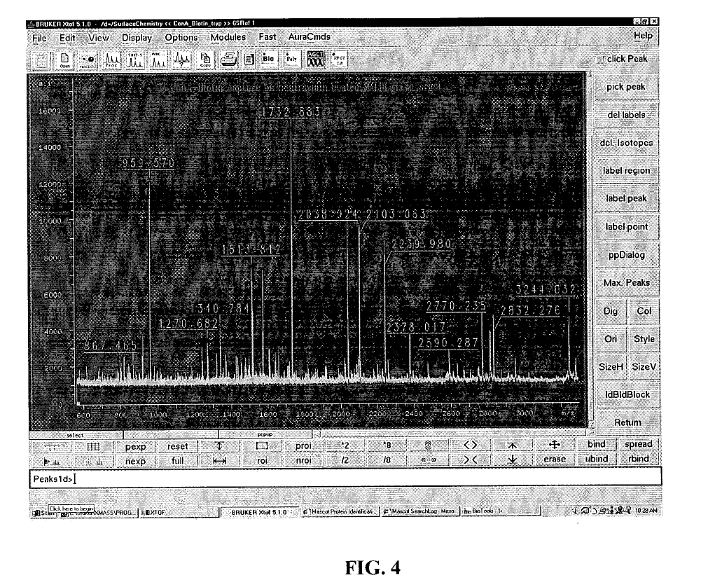

FIG. 5 shows Glutathione-S-Transferase-Biotin captured on Neutravidin coated MALDI glass target. Genetically engineered Schistosoma mansoni Glutathione-S-Transferase was expressed in E. coli. The Glutathione-S-Transferase was captured from a crude bacterial lysate on the MALDI target. E. coli proteins were removed with a mild washing procedure leaving a clean Glutathione -S-Transferase preparation as judged by the MALDI spectrum.

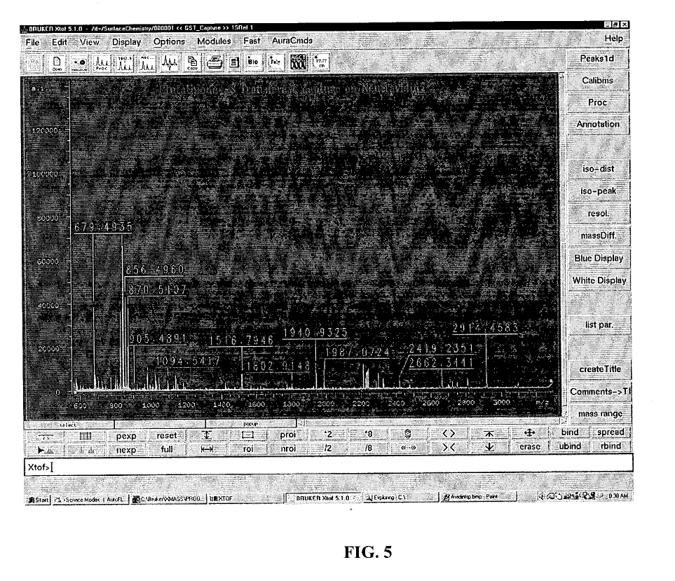

FIG. 6 shows spectrum of a Neutravidin digest analysed with a customized version of XMASS 5.1. The spectral analysis algorithm automatically detects peaks derived from Neutravidin and excludes them from the peaklist. This is a convenient method for automated peak detection on a protein array which is then used for the capture of proteins, metabolites or drug compounds. Peptide peaks derived from Neutravidin and from the captured biotinylated bait protein can be included in a database and are then automatically labeled in the spectra and excluded from the result peaklist.

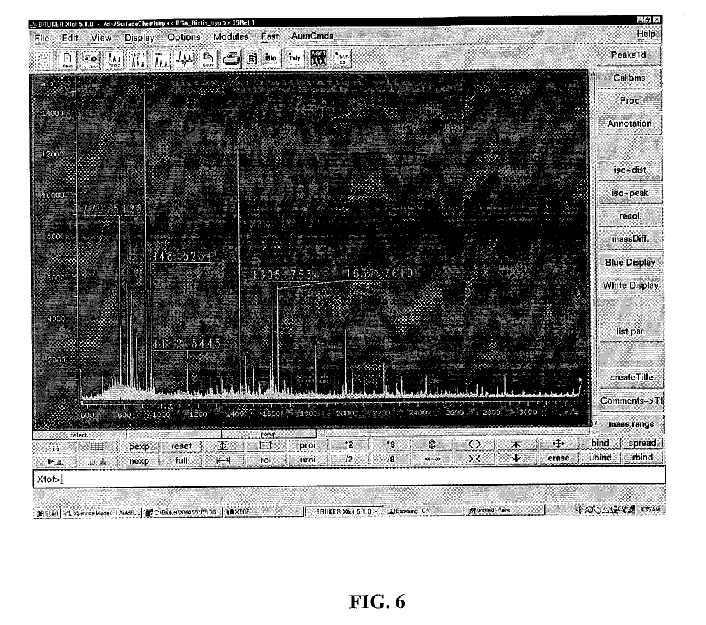

FIG. 7 shows random coupling and orientated coupling of proteins to a MALDI target.

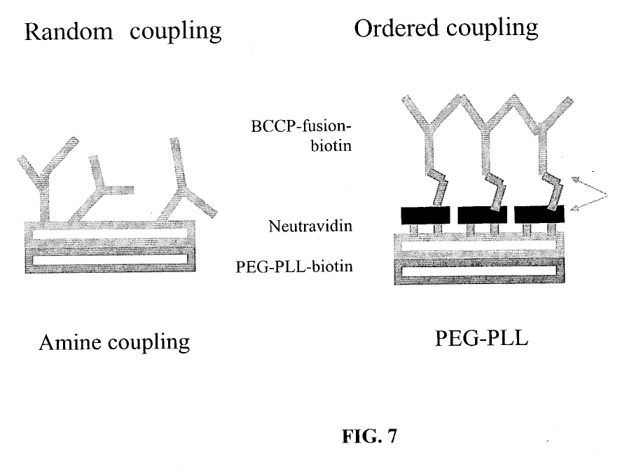

FIG. 8 shows a MALDI target loading kit with MALDI target.

FIG. 9 shows a MALDI target inserted into the adaptor bottom. The adaptor bottom can accommodate the MALDI target and aligns it in the right position for the adaptor top

FIG. 10 shows a MALDI target adaptor bottom, MALDI target and adaptor top assembled as seen from the top. From this top view the 16×24 holes in the adaptor top can be seen. In this assembled form each well can take 60 microlitres of liquid.

FIG. 11 shows a MALDI target adaptor bottom, MALDI target and adaptor top assembled shown from the side view. The side View of two supports from the adaptor bottom can be seen. The adaptor top is milled out on the opposite side to harbour the adaptor bottom. In total there are six supports from the adaptor bottom reaching to the adaptor top to give a stable support and to serve as a alignment means for the adaptor top. The MALDI target can be seen in between the adaptor as it forms a sandwich around it.

FIG. 12 shows a MALDI target adaptor for protein arrays.

Certain embodiments of the invention are described in the following examples, which are not meant to be limiting.

### EXAMPLES

### Material and Methods

Autoflex mass spectrometer, MALDI targets gold #26993 and glass #26754, Bruker Daltonics, Bremen, Germany.

Matrix: α-cyano-4-hydroxycinnamic acid, 2,5 Dihydroxybenzoic acid, Sinapinic acid, Lectin from Arachis hypogaea biotin labeled, Lectin from Lens culinaris biotin labeled, Concanavalin A biotin labeled, Albumin biotinamidocaprol labeled, Insulin biotin labeled, were purchased from Sigma, St. Louis, Mo. Glutathione S-transferase from Schistosoma mansoni was expressed in E. coli XL10 -Blue. Nitrogen: 99.9996 purity Linde, UK, TPCK treated Trypsin sequencing grade, Promega

Buffers and solutions: washing buffer: 1 mM Tris-HCl pH 7.5 with 0.1% Triton X-100, desalting buffer: 1 mM Tris-HCl pH 7.5.

### Example 1

### MALDI Target Surface Coating

MALDI targets are cleaned before use with acetone, acetonitrile, double distilled water and dried under nitrogen. Each position on the MALDI target is coated with 500 nanoliter 1%PLL-PEG-2%Biotin solution dissolved in desalting buffer. The target is then placed in a humid chamber for one hour at room temperature. Unbound PLL-PEG is rinsed with 200 ml washing buffer followed by two washes with 300 ml washing buffer. The target surface is dried under nitrogen. The PLL-PEG coating is then overlaid with 500 nanoliter of 0.5 mg/ml Neutravidin at each position of the array, incubated for one hour at RT in a humid chamber, rinsed with washing buffer, washed twice with 300 ml washing buffer and dried under nitrogen. THE MALDI target is now ready to be used as a highly specific affinity capture surface.

### Example 2

### Surface Capture of Biotinylated Macromolecules

A PLL-PEG-biotin neutravidin surface on a MALDI target is overlaid with 500 nanoliters of biotinylated protein for 2 hours and then washed twice with washing buffer followed by two washes with desalting buffer. Each sample on the target can be then overlaid with 500 nanoliters saturated α-cyano-4-hydroxycinnamic acid in acetone, or it can be further treated with trypsin for mass spectrometry fingerprint analysis or the protein array can be probed with small molecules, proteins, DNA or RNA.

### Example 3

### Tryptic Digestion on the MALDI Target

The protein coated MALDI target is dried under nitrogen and overlaid with a solution of 500 nanoliter 0.01 mg/ml trypsin in 25 mM ammoniumbicarbonate pH 7.5 and incubated in a humid chamber for 2 hours at 37° C., the MALDI target is then removed from the humid chamber and the solution is evaporated under nitrogen. The dried surface is then overlaid with a 500 nanoliters energy absorbing matrix molecules e.g. sinapinic acid, 2,5 Dihydroxybenzidine and α-cyano-4-hydroxzcinnamic acid.

### Example 4

### Mass Spectra Acquisition and Peak Annotation

Mass spectra are collected on a Bruker Autoflex MALDI TOF mass spectrometer. For different purposes the acquisition protocol and the use of the mass spectrometer hardware can be divided into two groups. When small molecule and tryptic peptide fragments are analyzed the MALDI TOF mass spectrometer is operated in the reflectron mode to achieve the highest possible mass resolution. For the analysis of high molecular weight molecules in the range of 10000 Da and higher the linear detection mode is chosen.

The experiments described in the examples herein, demonstrate that conventional glass and gold MALDI targets can be modified for the capture of native tagged functional proteins. Tryptic digestion and sample preparation were also possible on the same target. These experiments are the key steps for the preparation of MALDI compatible protein arrays.

The highly specific affinity capture of biotinylated proteins on surface coated MALDI targets opens new ways for sample preparation. The strong binding of the biotinylated protein to the surface coating allows vigorous washing steps including detergents to increase sample purity and furthermore samples can be effectively desalted before tryptic digestion. The handling of difficult biological samples is significantly improved by this method.

The Inventors have conducted experiments involving the capture of large numbers of human proteins on a single MALDI target thus providing novel methods for studying protein-protein, protein-DNA and protein-small molecule interactions.

### Example 5

### Scalable MALDI Target Sample Volume Loading Kit

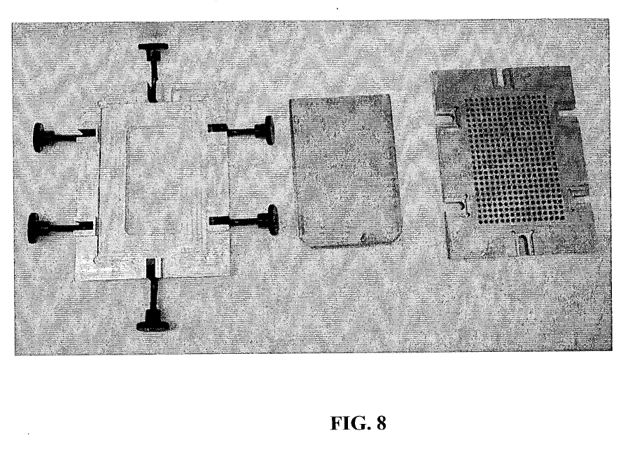

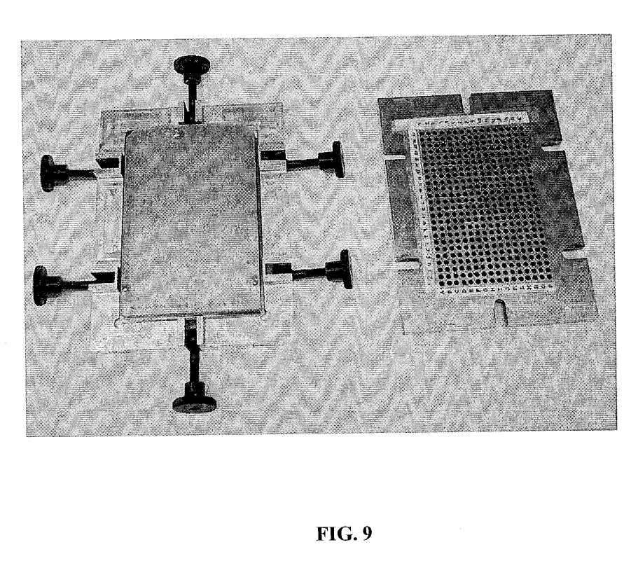

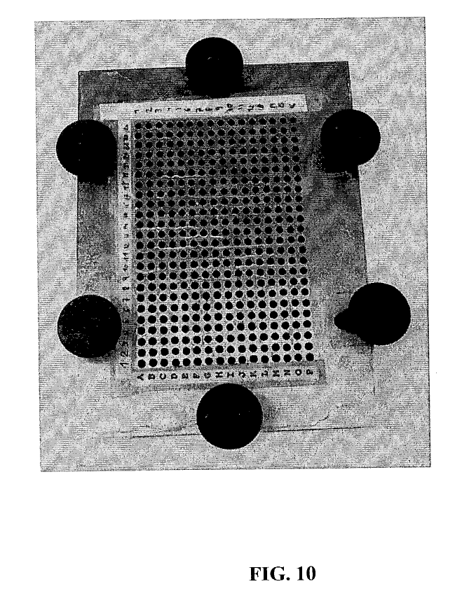

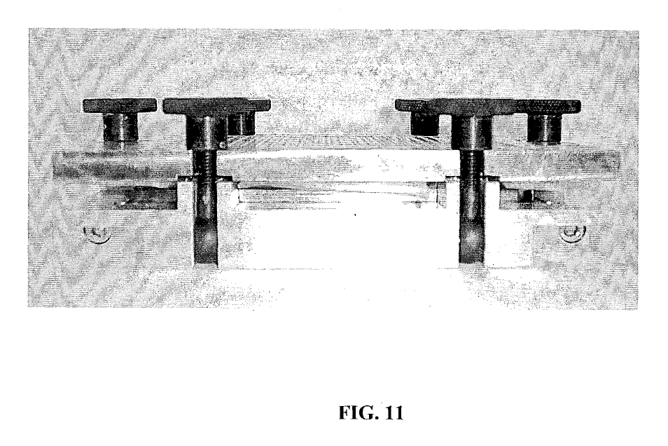

The adapter (see FIGS. 8 to 11) consists of two elements and incorporates the MALDI target in a sandwich arrangement. The bottom of the adapter forms a frame for proper alignment of the MALDI target. The adapter contains six flexible screw clamps with a hinge in the peripheral part. The MALDI target may be placed on the bottom of the adapter and can be covered by the adapter top plate. The top plate contains 384 holes, which forms 384 wells on top of the MALDI target surface, for example, a capture surface.

FIG. 8 shows a MALDI target loading kit with MALDI target. The figure shows from the left to the right the adapter bottom, MALDI target and adaptor top. The adaptor bottom has 6 tightening screws in the periphery, two on the right and two on the left side, as well as one on the top and bottom. The centre of the adaptor bottom is milled out to accommodate the MALDI target shown to right of the adaptor. The MALDI target is shown in between the adaptor bottom and top. The MALDI target is designed to accommodate 384 samples and the adaptor top has the same number of holes perfectly aligned with the MALDI target sample deposition areas. The adaptor top has six holes to fit in the tightening screws from the adaptor bottom. A 2 mm silicon layer is attached underneath the adaptor top. The silicon layer has 384 holes aligned with the adaptor top to allow liquid to reach the MALDI target and to seal each individual well to prevent leaking liquid from one well to the other.

### Example 6

### MALDI Target Adaptor For Protein Arrays

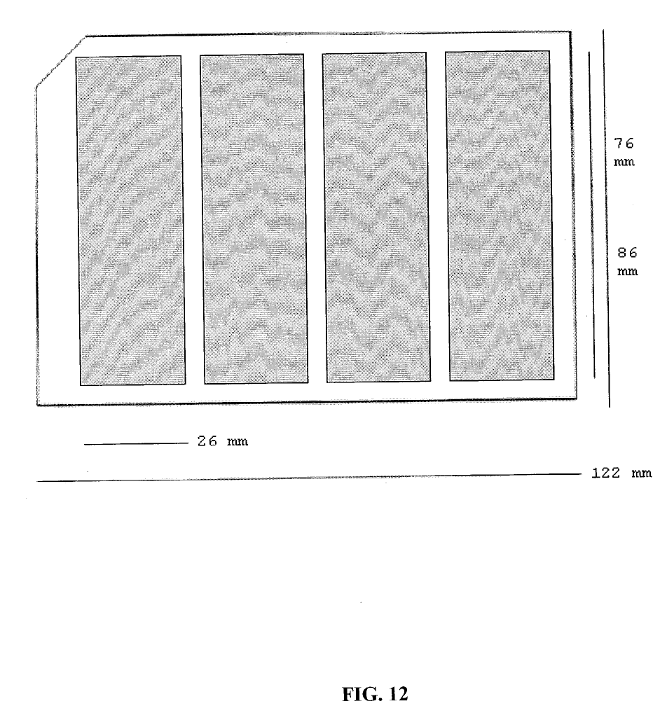

At present protein arrays are commonly printed out on microscope glass whereas mass spectrometer MALDI targets are of the size of 122×86 mm. The Inventors have developed an adaptor device suitable for use in the methods of the invention. The adapter (see FIG. 12) presents an interface to hold four glass microscope slides of 26×76 mm on a 122×86 mm Bruker Daltonics™ MALDI target. The microscope glass slide has a gold coating that confers conductivity to it. The glass slide is conductive and carries a protein resistant surface. The surface carries a capture surface, for example Neutravidin or Avidin or Streptavidin to capture biotinylated proteins, peptides, carbohydrates, DNA, RNA or non-biological homo-polymers or hetero-polymers

[image]

[image]

[image]

[image]

[image]

[image]

[image]

[image]

[image]

[image]

[image]

[image]

### References

Brian M. Austen, Emma R. Frears and Huw Davies. (2000) Journal of Peptide Science 6, 459-469

Maggie Merchant and Scot R. Weinberger. (2000) Electrophoresis 21, 1164-1167.

### Incorporation by Reference

The entire disclosure of each of the aforementioned patent and scientific documents cited hererinabove is expressly incorporated by reference herein.

### Equivalents

The invention can be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein.

I claim:

1. A probe for analysis of one or more proteins by laser desorption/ionisation mass spectrometry, wherein said proteins comprise a tag which in turn comprises a biotin group and wherein said probe comprises at least one surface comprising one or more streptavidin, avidin or neutravidin molecules that bind said biotin group to said surface.

2. The probe as claimed in claim 1 which further comprises one or more proteins carrying a tag bound via a biotin group to one or more streptavidin, avidin or neutravidin molecules present on the surface of said probe.

3. The probe as claimed in claim 1 or claim 2 wherein said one or more streptavidin, avidin or neutravidin molecules are present on the surface of the probe with a protein repellent coating on said surface.

4. The probe as claimed in claim 3 wherein said protein repellent coating is polyethylene biotin conjugated poly-L-lysine.

5. The probe as claimed in claim 4 wherein said tag is a BCCP tag or an Avi-tag (biotinylated peptide).

6. The probe as claimed in any one of claims 1 to 5 which comprises two or more proteins attached via a biotin group at known locations on the surface of the chip to form a protein array.

7. A method of analysis by laser desorption/ionisation mass spectrometry comprising the steps of:
a) providing a probe comprising at least one surface comprising one or more streptavidin, avidin or neutravidin molecules;
b) bringing said probe into contact with one or more proteins comprising a tag which in turn carries a biotin group under conditions allowing said biotin group to bind to at least one of said streptavidin, avidin or neutravidin molecules;
c) performing laser desorption/ionisation mass spectrometry on the proteins on the surface of the probe.

8. The method of claim 7 which comprises the alternative steps of:
b)i) bringing said probe into contact with one or more proteins comprising a tag which in turn carries a biotin group under conditions allowing said biotin group to bind to at least one of said streptavidin, avidin or neutravidin molecules;
b)ii) removing unbound molecules from the probe.

9. The method of claim 8 wherein said one or more proteins are contained in a mixture of tagged and untagged proteins.

10. The method of claim 7 or 8 which is a method for identifying a protein on the surface of the probe and which comprises the additional steps of:
d) determining the mass of the protein molecule
e) performing a digestion upon a replicate sample of said protein on a further probe or probe surface
f) performing laser desorption/ionisation mass spectrometry on the peptides resulting from step e) to identify said protein.

11. The method of claim 7 or 8 which is a method of analysing the function of a protein on the surface of the probe and a molecule interacting with said protein and which comprises the additional steps of:
c) bringing a protein on the probe surface into contact with one or more test molecules;
d) removing unbound test molecules from the probe surface;
e) performing laser desorption/ionisation mass spectrometry on the protein and any bound molecule to determine the identity of the protein and/or test molecule.

12. The method of claim 7 or 8 which is a method of analysing the function of a protein and which comprises the additional steps of:
c) bringing a protein on the probe surface into contact with one or more test substrates
d) performing laser desorption/ionisation mass spectrometry on the protein and test substrates to determine the presence and/or identity of products of catalysis of said test substrates by the protein.

13. The method of claim 7 or 8 which is a method of analysing the folded structure of a protein and which comprises the additional steps of:
d) determining the mass of the protein molecule;
e) performing hydrogen/deuterium exchange on solvent accessible amide groups on a replicate sample of said protein on a further probe or probe surface;
f) performing laser desorption/ionisation mass spectrometry on the protein resulting from step e) to determine the mass of said protein;
g) bringing the a replicate sample of said protein on a further probe or probe surface into contact with a denaturant to unfold said protein;
h) performing hydrogen/deuterium exchange on solvent accessible amide groups on the protein sample of step g) on the surface of the probe
i) performing laser desorption/ionisation mass spectrometry on the protein resulting from step h) to determine the mass of said protein;
j) comparing the information obtained from steps d), f) and i) to determine the folded structure of the protein.

14. The probe of any one of claims 1 to 6 or the method of any one of claims 7 to 13 wherein said probe carries a protein array.

15. The probe or method of claim 14 wherein said array consists of individual proteins present at at least 96, 384, 1536 or 10,000 discrete locations on said probe surface.

16. A MALDI target sample volume loading device comprising:
a sample loading means,
and
a MALDI target holding means,
wherein said sample loading means comprises a plurality of apertures suitable for loading samples on to a MALDI target (when present) which is held adjacent to one surface of said sample loading means by way of releasable engagement between said sample loading means and said MALDI target holding means.

17. The MALDI target sample volume loading device of claim 16 wherein said sample loading means and said MALDI target holding means releasably engage through interlocking features present on each means.

18. The MALDI target sample volume loading device of claim 16 or 17 wherein said plurality of apertures comprised by said sample loading means are present as a regularly spaced array of apertures.

19. The MALDI target sample volume loading device of claim 18 wherein said array of apertures comprises 384 apertures arranged in a 16×24 array.

20. The MALDI target sample volume loading device of any one of claims 16 to 19 wherein said sample loading means further comprises a liquid repellent layer on the surface adjacent to a MALDI target (when present).

21. The MALDI target sample volume loading device of claim 20 wherein said liquid repellent layer is a silicon layer.

22. The MALDI target sample volume loading device of any one of claims 16 to 21 which is shown in FIGS. 8, 9, 10 and 11.

23. The probe of any one of claims 1 to 6 or the method of any one of claims 7 to 15 wherein said probe is brought into contact with with one or more proteins comprising a tag by the use of a target sample volume loading device according to any one of claims 16 to 22.

24. The method of any one of claims 8 or 10 to 16 wherein said alternative steps or additional steps are carried out in one or more wells formed by a target sample volume loading device according to any one of claims 16 to 22.

25. A MALDI target adaptor device suitable for locating one or more glass microscope slides on a MALDI target (when present), wherein said glass slides comprise a conductive coating, a protein resistant surface and a capture surface present on said protein resistant surface.

26. The MALDI target adaptor device of claim 25 wherein said conductive coating is gold.

27. The MALDI target adaptor device of claim 25 or 26 wherein said capture surface comprises neutravidin, avidin or streptavidin.

28. The MALDI target adaptor device of any one of claims 25 to 27 which is suitable for locating four glass microscope slides on a MALDI target.

29. The MALDI target adaptor device of claim 28 wherein said MALDI target is a Brucker Daltonics™ MALDI target.

30. The MALDI target adaptor device of any one of claims 25 to 29 which is shown in FIG. 12.

31. The probe of any one of claims 1 to 6, 23 or 24 or the method of any one of claims 7 to 15, 23 or 24 wherein said probe is mounted on a MALDI target adaptor device according to according to any one of claims 25 to 30.
